(19) United States
(12) Patent Application Publication
(10) Pub. No.: US 2007/0004969 A1
(43) Pub. Date: Jan. 4, 2007

(54) Health monitor

(75) Inventors: Yuan Kong, Kirkland, WA (US); David Joshua Kurlander, Seattle, WA (US); Silviu-Petru Cucerzan, Redmond, WA (US); Zhengyou Zhang, Bellevue, WA (US); David W. Williams, Woodinville, WA (US); Zicheng Liu, Bellevue, WA (US); Michael J. Sinclair, Kirkland, WA (US)

Correspondence Address: AMIN. TUROCY & CALVIN, LLP, 24TH FLOOR, NATIONAL CITY CENTER, 1900 EAST NINTH STREET, CLEVELAND, OH, 44114, US

(73) Assignee: Microsoft Corporation, Redmond, WA (US)

(21) Appl. No.: 11/170,525

(22) Filed: Jun. 29, 2005

(51) Int. Cl. A61B 5/00

(52) U.S. Cl. 600/300; 128/920

(57) ABSTRACT

A device and/or devices can be placed on an individual to continuously collect and/or transmit information regarding the health state of the individual. The monitor, for example, can transmit data to doctor(s), hospital(s), emergency personnel, as well as automatically initiate affirmative actions (e.g., call an ambulance). The invention can facilitate preventive medicine by monitoring the individual's day to day actions (e.g., eating habits, exercise, stress, sleep, allergy detection, heart rate, blood sugar). This monitored information can be employed to facilitate the individual in taking proactive steps to living a healthy life. The device can also be used to record a user reaction to an event and/or to broadcast information about oneself whether for health, business or social purposes. Additionally, the invention can be used as a driver monitor to facilitate safe handling of equipment (e.g., alcohol sensor).

### BACKGROUND

Both enterprises and individuals are increasingly interested in using handheld devices. Most modern handheld devices are equipped with multiple sensors (e.g., microphone, wireless transmitter, global positioning system (GPS) engine, camera, stylus, etc.). However, there are no applications available that make full use of multiple sensors. In other words, multi-sensory technologies that make handheld devices a multi-modal multi-lingual mobile assistant are not available.

Physiological sensors can be employed to measure and supply data related to a host of factors associated with the health state of an individual. For example, physiological monitors (e.g., sensors) are widely available to measure criterion such as blood pressure, heart rate, blood sugar and even brain waves. Physiology can be divided into numerous subdivisions. For example, electrophysiology refers to the operation of the nerves and systems thereof together with muscles. Neurophysiology refers to the physiology of the brain. Furthermore, cell physiology refers to the functioning of individual cells. Monitors can be employed to measure criteria related to any subdivision of physiology.

The environment can affect the health state of an individual. For example, high pollen levels can greatly affect an individual that suffers from a pollen allergy. Similarly, heat and sun (as well as cold temperatures) can greatly affect the health state of an individual. Although these examples are related to nature, other man-made environmental factors can affect the health state of an individual. By way of example, second-hand smoke, friable asbestos, silica dust, as well as other forms of air-pollution, have been proven to inhibit the quality of air taken in by humans. Oftentimes, these factors can lead to the development of cancer by the individual.

In accordance with a physiological criterion as described above, these environmental factors can also be monitored. By way of example, pollen levels and pollution indices are just two frequently measured environmental conditions. These conditions are frequently disseminated via newscasters and meteorologists. As well, beach forecasts often include a rating of the intensity of the sun for a given day.

Today, cellular telephones running on state-of-the-art operating systems have increased computing power in hardware and increased features in software in relation to earlier technologies. For instance, cellular telephones are often equipped with built-in digital image capture devices (e.g., cameras) and microphones together with computing functionalities of personal digital assistants (PDAs). Since these devices combine the functionality of cellular phones with the functionality of PDAs, they are commonly referred to as “smartphones.” The hardware and software features available in these smartphones and similar technologically capable devices provide developers the capability and flexibility to build applications through a versatile platform.

The popularity of the emerging technologies of these portable devices such as cellular telephones, smartphones and PDAs continues to increase. As illustrated above, the functionality and capabilities of these devices continues to evolve. For example, cameras and personal music players have become commonplace in many handheld devices.

To this end, conventional portable devices do not communicate with and/or include physiological and/or environmental sensors. As well, today's devices do not combine physiological criterion together with environmental criterion to determine a health state of an individual. For example, an evaluation of the user's heart rate does not conventionally consider environmental factors, such as the speed of the user (e.g., if the user is running, walking, or laying in bed), the humidity of the air, the altitude, etc.

### SUMMARY

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

The invention disclosed and claimed herein, in one aspect thereof, comprises a device and/or devices that can be placed on or near an individual to continuously collect and/or transmit information related to the health state of the individual. The monitor, for example, can transmit data to doctor(s), hospital(s), emergency personnel, as well as automatically take proactive action (e.g., call an ambulance).

The functionality of the invention can be employed within a stand-alone component as well as incorporated into a portable device having other core functionality. In one aspect, the monitoring functionality can be integrated into a cellular telephone or personal data assistant (PDA). Additionally, other aspects exist whereby the novel functionality of the invention can be retrofitted into an existing handheld device.

In yet another aspect, the invention can facilitate preventive medicine by monitoring day-to-day actions (e.g., eating habits, exercise, stress, sleep, allergy detection, heart rate, blood sugar) of an individual. This monitored information can be employed to ensure the individual is taking proactive steps to living a healthy life.

In addition to physiological effects, the invention can also be used to record a user reaction to an event and/or to broadcast information about oneself whether for health, business or social purposes. For example, an image capture system and/or microphone can be employed with physiological sensors to associate (and memorize) a user reaction to an event.

In still another aspect, the invention can be used as a driver monitor to facilitate safe handling of equipment (e.g., alcohol sensor). Health states can be monitored and/or analyzed at any desired frequency or sampling rate. These recorded health states can facilitate functionality analogous to a “black box recorder” thereby enabling a recreation of events leading to an injury or even death. This historical playback can also be used to promote healthy living.

In yet another aspect thereof, an artificial intelligence component is provided that employs a statistical analysis to prognose or infer an action that a user desires to be automatically performed. As well, rules-based logic components can be employed to set or predetermined thresholds, benchmarks and/or actions that effect automating functionality.

To the accomplishment of the foregoing and related ends, certain illustrative aspects of the invention are described herein in connection with the following description and the annexed drawings. These aspects are indicative, however, of but a few of the various ways in which the principles of the invention can be employed and the subject invention is intended to include all such aspects and their equivalents. Other advantages and novel features of the invention will become apparent from the following detailed description of the invention when considered in conjunction with the drawings.

### BRIEF DESCRIPTION OF THE DRAWINGS

FIG. 1 illustrates a general component block diagram of a system that employs a sensing component and an analyzer component in accordance with an aspect of the subject invention.

FIG. 2 illustrates an exemplary flow chart of procedures that analyze and process a sensor output in accordance with a disclosed aspect.

FIG. 3 illustrates a general component block diagram of a portable device having an environmental component and a physiological sensor component in accordance with an aspect of the subject invention.

FIG. 4 illustrates a general component block diagram of a system that includes a communication component that facilitates data transmission in accordance with an aspect of the subject invention.

FIG. 5 illustrates a general component block diagram of a system having a recording component and optional data store in accordance with an aspect of the subject invention.

FIG. 6 is a portable device that can employ multiple environmental sensors and multiple physiological sensors in accordance with an aspect of the invention.

FIG. 7 illustrates a graphical representation of an individual having multiple sensors associated therewith in accordance with an aspect of the invention.

FIG. 8 is a schematic block diagram of a portable handheld device according to one aspect of the subject invention.

FIG. 9 illustrates an architecture of a portable handheld device including an artificial intelligence-based component that can automate functionality in accordance with an aspect of the invention.

FIG. 10 illustrates an architecture of a portable handheld device including a rules-based logic component that can automate functionality in accordance with an aspect of the invention.

FIG. 11 illustrates a block diagram of a computer operable to execute the disclosed architecture.

FIG. 12 illustrates a schematic block diagram of an exemplary computing environment in accordance with the subject invention.

### DETAILED DESCRIPTION

The invention is now described with reference to the drawings, wherein like reference numerals are used to refer to like elements throughout. In the following description, for purposes of explanation, numerous specific details are set forth in order to provide a thorough understanding of the subject invention. It may be evident, however, that the invention can be practiced without these specific details. In other instances, well-known structures and devices are shown in block diagram form in order to facilitate describing the invention.

As used in this application, the terms “component” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

As used herein, the term to “infer” and “inference” refer generally to the process of reasoning about or inferring states of the system, environment, and/or user from a set of observations as captured via events and/or data. Inference can be employed to identify a specific context or action, or can generate a probability distribution over states, for example. The inference can be probabilistic—that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources.

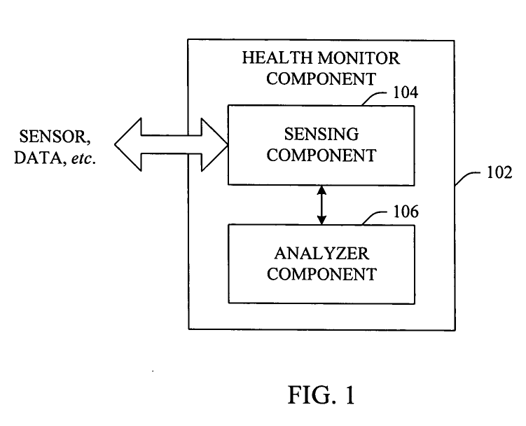

Referring initially to FIG. 1, a health monitor component 102 in accordance with an aspect of the invention is illustrated. Generally, health monitor component 102 can include a sensing component 104 and an analyzer component 106. As will be described in more detail infra, sensing component 104 can obtain a health related input. The following scenarios, and other scenarios described hereinafter, are provided merely to provide context to the invention. These scenarios are not intended to limit the novel functionality of the invention and/or scope of the claims appended hereto. Accordingly, these and other scenarios are to be included in the scope of this disclosure and claims appended hereto.

In a first scenario, suppose an individual has a history of high blood pressure. The health monitoring component 102, and more specifically the sensing component 104, can be employed to receive an input representing a blood pressure reading from a local or remote sensor. As illustrated by the bi-directional arrow, the sensing component can receive and/or detect information and data. In another scenario, the sensor (e.g., blood pressure monitor) can be internal to the health monitor component 102. This exemplary aspect will be described in greater detail infra.

In another scenario, the sensing component 104 can include and/or integrate with an environmental sensing component. For example, the health monitor component 102 can include an environmental sensor that can effect reading an environmental (e.g., ambient) condition relative to a location of the health monitor component 102. In disparate aspects, the sensing component 104 can measure ambient temperature, motion, altitude, pollution index, pollen level, etc. It is to be appreciated that the invention can include multiple sensing components (e.g., 104) thus multiple readings can be obtained.

Analyzer component 106 can, in turn, evaluate the sensor reading whereby a health state can be established. In one example, the sensor reading can be evaluated based upon a predetermined threshold and/or benchmark value. In another aspect, the sensor reading can be evaluated based upon a disparate sensor reading(s). As will be described in detail infra, artificial intelligence (AI) and/or rules-based logic can be employed to intelligently effect evaluation of the sensor input(s).

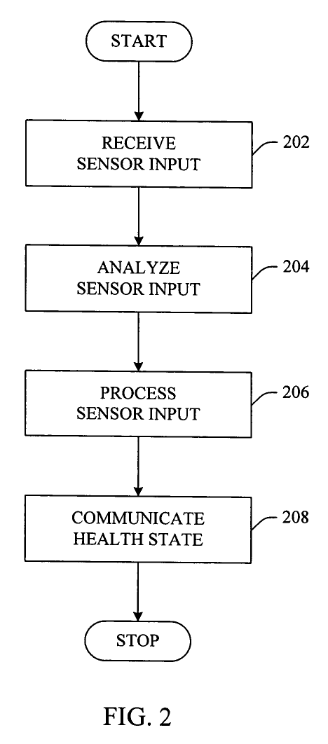

FIG. 2 illustrates a methodology of processing a sensor input in accordance with an aspect of the invention. While, for purposes of simplicity of explanation, the one or more methodologies shown herein, e.g., in the form of a flow chart, are shown and described as a series of acts, it is to be understood and appreciated that the subject invention is not limited by the order of acts, as some acts may, in accordance with the invention, occur in a different order and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with the invention.

At 202, an input is received. As described supra, this input can be received via a physiological sensor or an environmental sensor component. In another aspect, this input can be inferred via AI or retrieved from a data store. Additionally, the input can be received (e.g., via external sensors) or fetched (e.g., via internal sensors) in alternative aspects of the invention.

At 204, the sensor input is analyzed and processed at 206. The sensor input can be evaluated based at least in part on a predetermined threshold, a statistical benchmark, input from another sensor or the like. Accordingly, at 204, 206 a health state can be established based upon applicable factors and criteria.

In one example, assume that a blood pressure monitor (e.g., sensor) detects a reading of 110/70. As will be understood, the first number is representative of the systolic blood pressure reading which is the maximum pressure exerted when the heart contracts. The second number is representative of the diastolic blood pressure reading which is the pressure of blood in the arteries when the heart is at rest.

Once received, the blood pressure reading can be processed to establish a health state of the individual. For example, the instant blood pressure reading can be compared to historical data, threshold data, statistical data or the like to determine the health state. At 208, this health state information can be communicated to an external entity. For example, the health state can be communicated to a recording component whereby a historic compilation of health states can be maintained. In another example, the health state can be communicated to a medical professional and/or medical facility for further analysis and/or action. In still another example, in the case of an emergency, an emergency service can be contacted at 208 in accordance with the sensor input. In accordance therewith, a language translator and/or converter can be provided whereby the communication can be translated and/or converted into a language comprehendible by a recipient. In other aspects, location detection systems and/or ambient sensors can be employed to identify an appropriate language for translation and/or conversion.

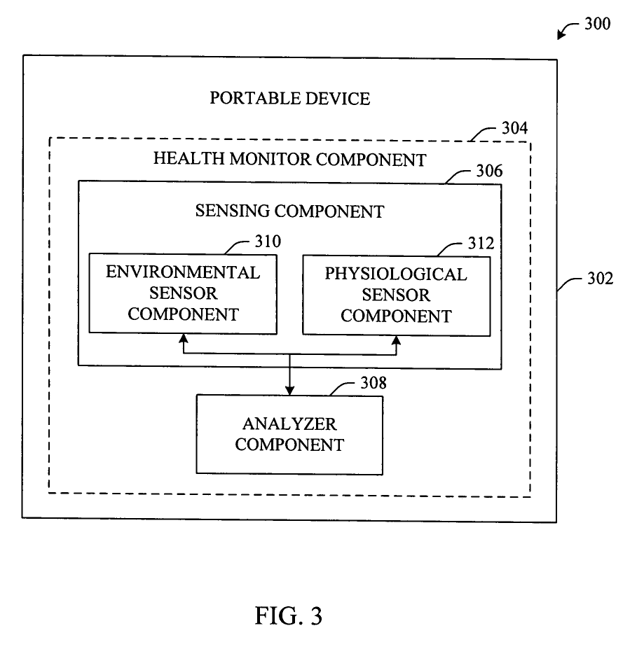

FIG. 3 is a block diagram of a system 300 that illustrates a portable device 302 having a health monitor component 304 integrated therein. It is to be appreciated that health monitor component 304 can be permanently integrated into or removable from the portable device 302. As such, in accordance with alternative aspects, health monitoring component 304 can be optionally integrated (e.g., retrofitted) into an existing portable device 302.

Portable device 302 can be any handheld device capable of integrating the health monitor component 304. In one example, portable device 302 is a cellular telephone. In another aspect, portable device 302 is a smartphone. In still other exemplary aspects, portable device 302 is a personal data assistant (PDA) or personal pocket computer (PPC). Although, the aspects described herein are directed toward interfacing health monitor component 304 into a portable device 302 having communications functionality in addition to the scope of functionality of the health monitor component 304 (e.g., cellular telephone), it is to be understood that the novel functionality of establishing health-related events and/or states in accordance with physiological and/or environmental effects can be integrated into a stand-alone component. In other words, in other aspects, the primary or sole functionality of portable device 302 can be the health-related functionality described herein.

As illustrated in FIG. 3, a sensing component 306 can include an environmental sensor component 310 and a physiological sensor component 312. Each of these sensors (310, 312) can affect measuring or detecting a criteria or condition. Moreover the sensor components (310, 312) can convert the measured and/or detected information into an analog or digital representation as appropriate. For example, an optical sensor can detect an intensity or brightness of light. In another example, environmental sensor component 310 can establish a detection of an environmental condition relative to the portable device 302. By way of even further example, the environmental sensor component 310 can measure pollen levels in a proximate area relative to the portable device 302.

As will be described in greater detail infra, global positioning satellite (GPS) systems and/or accelerometers can be employed to determine if the location of the portable device changes such that a new pollen level rating would be in order. This determination can be made based upon any number of factors including, but not limited to, relative distance from a previous reading, ambient temperature and/or moisture changes or the like. In other aspects, AI and/or rules-based logic can be employed to determine if a new pollen level reading or other measurement should be taken. Alternatively, the system can continuously monitor a relevant factor (e.g., pollen level) at a pre-determined sampling rate.

The physiological sensor component 312 can be employed to monitor any physiological criteria desired. For example, the physiological sensor component 312 can be employed to monitor electrophysiological, neurophysiological and cellular physiological conditions. The physiological sensor component 310 together with the environmental sensor component 310 can be employed to monitor ecophysiological effects related to an individual. In other words, the physiological sensor component 312 together with the environmental component 310 can be employed to determine how ecology affects the physiological traits of an individual and vice-versa. For example, dehydration can occur in humans during elevated physical activity. It will be understood that this dehydration condition can be enhanced and/or affected by environmental conditions.

Analyzer component 308 can be employed to process the outputs of the sensor components (310, 312). Accordingly, analyzer component 308 can establish a health state which can later be acted upon as described infra. Additionally, the analyzer component 308 can facilitate establishing a health history (e.g., chronology of health states) that can be used to promote healthy living, health-related care, retrospective analysis of a lifestyle of an individual, etc.

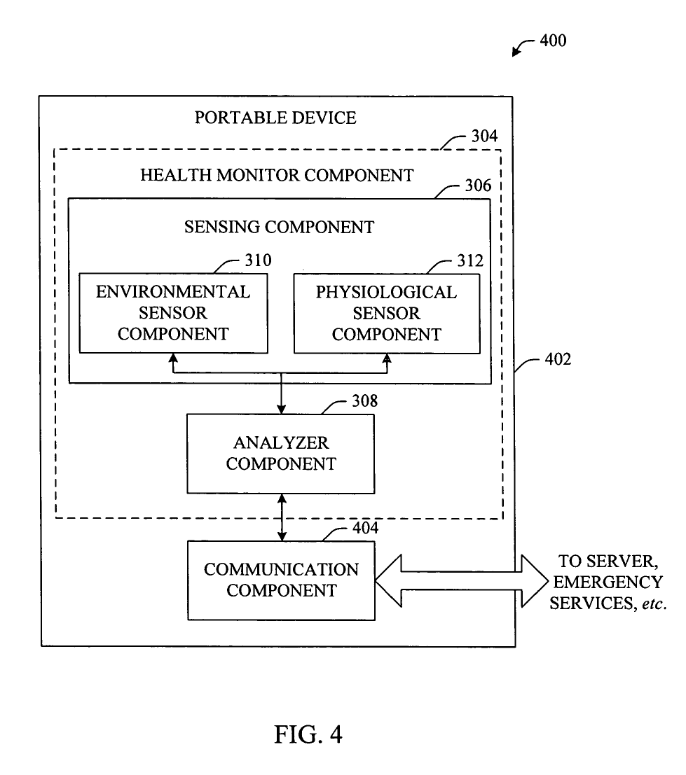

Referring now to FIG. 4, a block diagram of system 400 in accordance with an aspect of the invention is shown. Generally, system 400 can include a portable device 402 having a health monitor component 304 integrated therein. As described supra, health monitor component 304 can be an expansion component/module thus being capable of retrofit into an existing portable device 402. As well, health monitor component 304 can optionally be removed from the portable device 402.

Health monitor component can include a sensing component 306 and an analyzer component 308. As described with reference to FIG. 3, sensing component 306 can include an environmental sensor component 310 and a physiological sensor component 312. Although the aspect shown illustrates a single environmental sensor component 310 and a single physiological sensor component 312, it is to be appreciated that a novel feature of the invention is the ability to employ multiple disparate sensor technologies. To this end, it is to be understood that alternative aspects can exist whereby additional sensors can be employed to provide input to the analyzer component 308.

The analyzer component 308 can receive inputs from the sensors (310, 312) and effect evaluation and manipulation of the data received. Accordingly, the analyzer component 308 can facilitate generation of a health state with respect to the input(s). Alternatively, the analyzer component 308 can pass through the data without any manipulation. This pass-through can be particularly useful in a data recordation scenario. It is to be understood that sensor components 310, 312 can employ any suitable wired and/or wireless protocol to transmit sensor outputs to the analyzer component 308.

System 400 can further include a communication component 404. The output of the analyzer component 308 can be transmitted to a remote location. In one example, the output (e.g., sensor reading, health state) can be transmitted to a remote server via the Internet. In another example, the output can be transferred to an emergency service (e.g., emergency medical technician). For example, when an individual is injured, the individual can transmit a picture or video of the wound to a remote emergency service thus an emergency medical technician can analyze the situation and provide advice/instructions. In accordance with aspects of the invention, communication component 404 can employ various wired and/or wireless protocols to transfer the output.

Additionally, the subject invention can employ language translation and/or conversion to effect comprehendible communication to a recipient. By way of example, a GPS system can be employed to determine an appropriate language and/or dialect of location of the portable device 402 or target transmission location. Accordingly, text and/or speech communication can be translated and/or converted to effect comprehendible communication with a recipient. This novel functionality is to be included in the scope of this disclosure and claims appended hereto.

The communication component 404 can employ a cellular network, Wi-Fi network, or the like to communicate to a remote entity. In another aspect, a wired connection can be employed. As the output of the analyzer component can be a computed (e.g., evaluated) health state as well as raw sensor data, it is to be appreciated that communication component 404 can transmit computed or raw data (or any combination thereof).

For example, in order to compile a health history, the communication component 404 can transmit a health state, or sequence of states, generated via analyzer component 308 to a remote server for compilation. Over time, chronologically stored health states can facilitate telling a life story of an individual. Effectively, this functionality can be viewed as analogous to a “black box recorder” applied to an individual. It is also to be appreciated that the functionality described herein can be applied to animals as well.

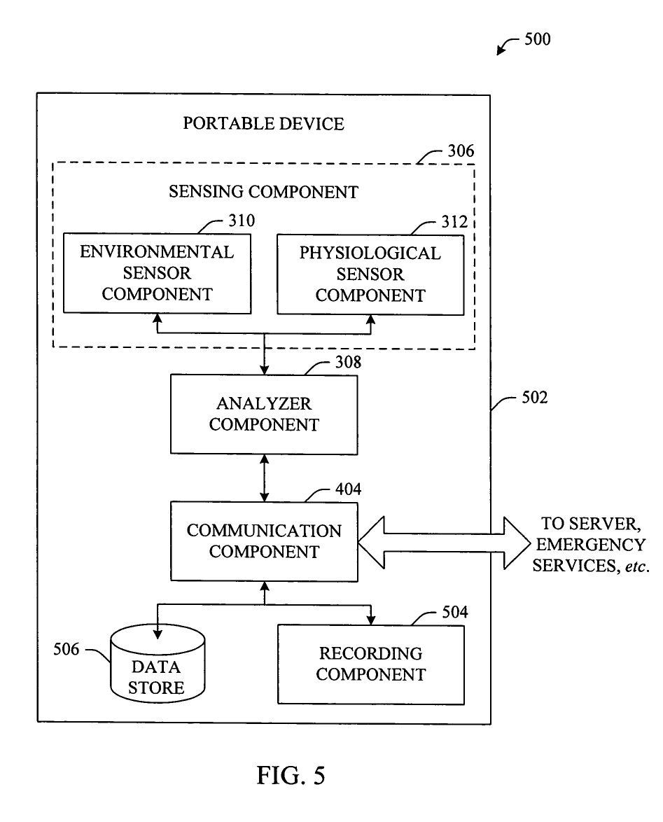

Referring now to FIG. 5, an alternate aspect of the invention is shown and represented by system 500. As illustrated, portable device 502 can include a sensing component 306, an analyzer component 308 and a communication component 404. As described with reference to FIGS. 3 and 4, sensing component 306 can include an environmental sensor component(s) 310 and/or a physiological sensor component(s) 312. In accordance therewith, analyzer component 308 can receive the output of the sensor components (310, 312), manipulate if desired, and transfer the data (e.g., raw or manipulated) to the communication component 404. Communication component 404 can transfer the data to a remote server, emergency service, or other desired location or entity.

Portable device 502 can additionally include a recording component 504 and a data store 506. Recording component 504 can facilitate locally recording the output of the analyzer component 308 into an on-board memory or data store 506. As described with reference to the remote storage device, on-board data store 506 can facilitate establishment of a historical representation of the health of an individual. For example, this representation can include a time-stamped chronology of health states of an individual. As well, since environmental sensor component 310 can capture proximate events and data (e.g., noise, temperature, air quality, . . . ) this chronology can be employed to proactively initiate and effect healthy living.

Further, as multiple sensors (310, 312) can be employed in accordance with the invention, the invention can monitor and alert of imminent health risks. By way of example, suppose an individual is allergic to insect bites/stings. Accordingly, anaphylaxis, a severe, whole-body allergic reaction, can result if this individual is stung by a bee. After an initial exposure to a substance like bee sting toxin, the immune system can become sensitized to the allergen. On a subsequent exposure, an allergic reaction can occur. This reaction can be sudden, severe, and even life threatening involving the whole body.

In this situation, tissues in different parts of the body release histamine and other substances. This causes constriction of the airways, resulting in difficulty in breathing, as well as gastrointestinal symptoms such as abdominal pain, cramps, vomiting, and diarrhea. These released substances as well as physiological symptoms can be monitored via sensors 310, 312. Additionally, sensors 310, 312 can monitor the effects of the release of histamine on blood pressure and/or volume. It is known that histamine can cause the blood vessels to dilate thereby lowering blood pressure. As well, histamine can cause fluid to leak from the bloodstream into the tissues thereby lowering the blood volume. Ultimately, if not monitored and detected in time, these effects can result in the individual going into shock. Other medical symptoms (e.g., hives) can occur as a result of this allergic reaction.

This exemplary scenario illustrates one novel feature and benefit of the invention. For example, in accordance with the invention, sensor component(s) 312 can detect release of histamine into the blood stream. As well, sensor component(s) 312 can detect a drop in blood pressure as well as a decrease in blood volume. Accordingly, the analyzer component 308 can evaluate the situation and determine if the detected limits are of concern.

If the analyzer component determines that the present condition is life threatening, the communication component 404 can be employed to alert an emergency service as shown. In any case, the histamine level in relation to blood pressure, blood volume and artery dilation can be recorded in data store 506 or transmitted to an external data store or server. This information can be later retrieved as desired. Additionally, this information can be employed by the analyzer component 308 in subsequent similar situations in order facilitate evaluations.

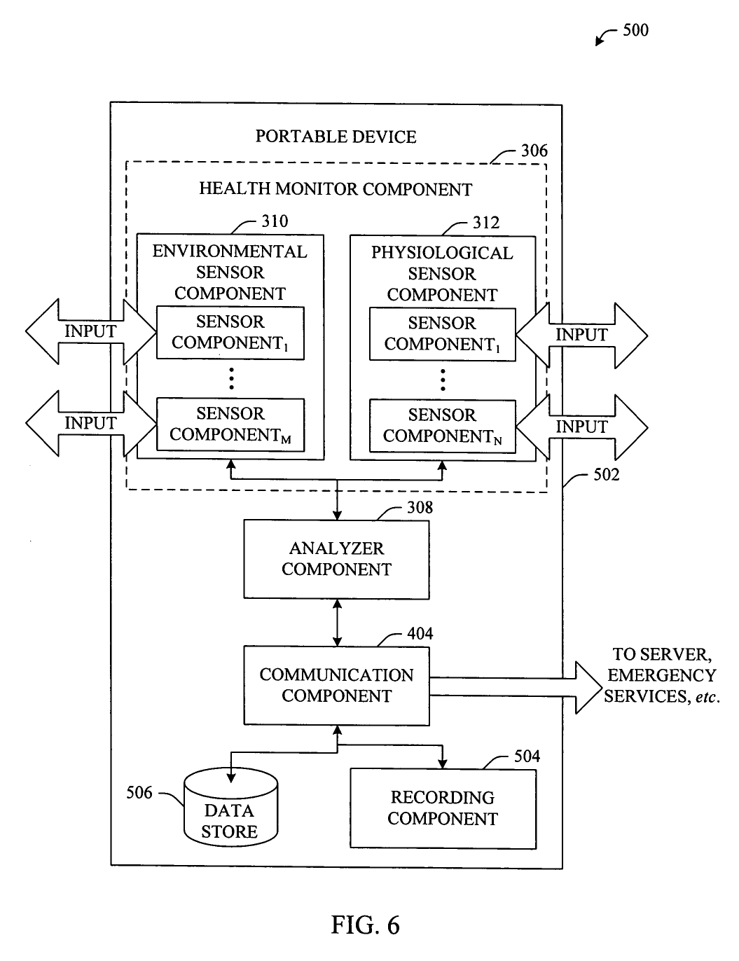

Turning now to FIG. 6, an alternative view of system 500 is shown. In particular, FIG. 6 illustrates that environmental sensor component 310 can include 1 to M sensor components, where M is an integer. As described supra with reference to FIG. 3, 1 to M environmental sensor components can be referred to individually, or collectively, as environmental sensor components 310.

Similarly, physiological sensor component 312 can include 1 to N sensor components, where N is an integer. As described supra with reference to FIG. 3, 1 to N physiological sensor components can be referred to individually, or collectively, as physiological sensor components 312.

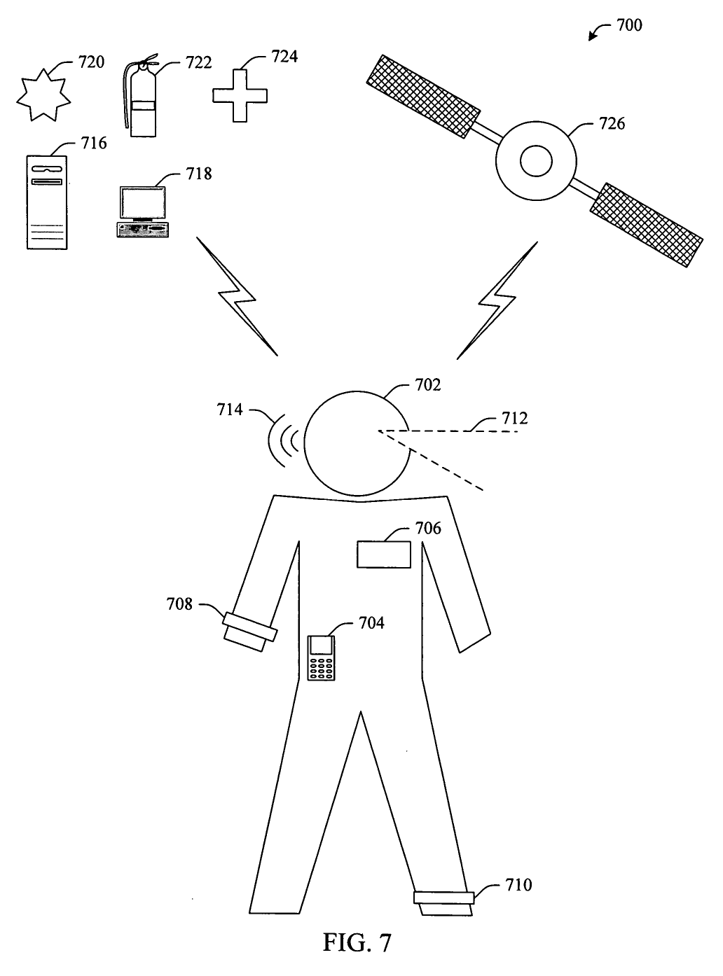

FIG. 7 illustrates a graphical representation of a system 700 that depicts numerous aspects and corresponding novel functionality of the invention. Generally, system 700 can include an individual 702 having a portable device 704 in relatively close proximity. As illustrated, portable device 704 can be attached to a waist belt area of user 702. It will be appreciated that the exemplary placement of portable device 704 can accommodate sensor communication via wireless personal area network (PAN) technologies such as Bluetooth™, infrared or the like.

System 700 can additionally include sensors (706, 708, 710). For example, a chest-mounted sensor 706 can be employed to monitor and detect a heart rate of individual 702. Similarly, wrist-mounted (e.g., wrist watch) sensor 708 can alternatively be employed to monitor pulse of the individual 702. Sensor 710 is shown to illustrate that additional sensors can be employed to monitor and/or detect any known criteria. Although sensors 706, 708, 710 are external to portable device 704, it is to be appreciated that the sensor functionality can be integrated into the portable device 704. As well, in accordance with alternate aspects, sensors implanted internal to individual 702 can be employed without departing from the scope and novel functionality of the invention described herein.

Other sensory perceptions can be monitored in accordance with the invention. By way of example, it will be appreciated that image capture devices are frequently integrated into portable devices (e.g., 704) to capture a visual representation of what an individual sees (represented by dashed lines 712). As such, an image capture device (not shown) can be employed to record a proximate event in relation to a physiological event. Therefore, the invention (via analyzer component) can effectively re-create the cause of a change in a physiological state.

Similarly, a microphone integrated into the portable device 704 can record audible sounds (e.g., snoring, water sounds, human voices, animal sounds (e.g., barking), horn sound) (as illustrated by 714) relative to a proximate event in relation to a change in physiological state. Accordingly, this recordability can facilitate a re-creation of a cause as related to change in physiological state. Additionally, the microphone can be employed to accept voice commands and information. A speech recognition engine and speech-to-text converter can be employed to interpret audible commands and thereby effect appropriate action. Moreover, the portable device 704 can include a text-to-speech engine that can be employed to audibly convey information and alerts. Additionally, a language converter can be employed to convert audible and/or text commands and/or information into a language comprehendible to a recipient.

Other environmental sensors can be employed in connection with portable device. By way of further example, a sensor can be integrated into portable device 704 whereby pollution, smoke and other air quality factors can be detected and/or monitored. It will be appreciated that these environmental sensors can effectively monitor, detect and rate the intensity and levels of each corresponding criteria. These levels can be employed by the analyzer component to establish a health state.

As previously described and illustrated in FIG. 7, portable device 704 can transmit data via wired and/or wireless protocols. In one example, the system can employ cellular network technology to transmit data (e.g., health state(s)) to a remote server 716 or personal computer 718. Additionally, as illustrated, portable device 704 can communicate to emergency services such as, police 720, fire 722, medical 724 or the like. The text-to-speech engine, speech-to-text engine and/or language converter can be employed to convey information and data. As well, portable device 704 can be equipped to communicate with a GPS system 726 whereby, a location of the device can be determined and/or transmitted. This GPS determined location can further be employed to determine a corresponding language(s) and/or dialects of a current location of the user 702 or entity for which the device 704 is communicating.

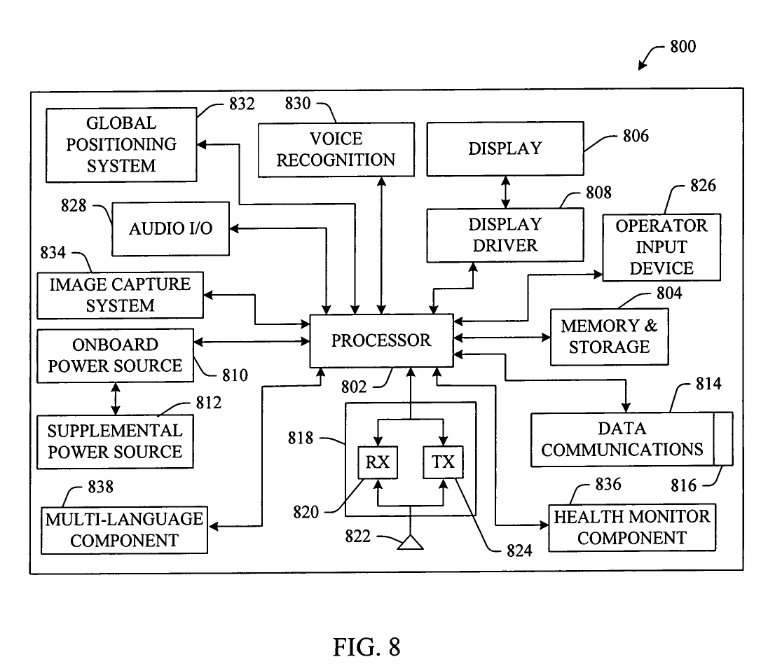

Referring now to FIG. 8, there is illustrated a schematic block diagram of a portable hand-held device 800 according to one aspect of the subject invention, in which a processor 802 is responsible for controlling the general operation of the device 800. The processor 802 can be programmed to control and operate the various components within the device 800 in order to carry out the various functions described herein. The processor 802 can be any of a plurality of suitable processors. The manner in which the processor 802 can be programmed to carry out the functions relating to the subject invention will be readily apparent to those having ordinary skill in the art based on the description provided herein.

A memory and storage component 804 connected to the processor 802 serves to store program code executed by the processor 802, and also serves as a storage means for storing information such as sensor outputs, health states or the like. The memory 804 can be a non-volatile memory suitably adapted to store at least a complete set of the information that is acquired. Thus, the memory 804 can include a RAM or flash memory for high-speed access by the processor 802 and/or a mass storage memory, e.g., a micro drive capable of storing gigabytes of data that comprises text, images, audio, and video content. According to one aspect, the memory 804 has sufficient storage capacity to store multiple sets of information, and the processor 802 could include a program for alternating or cycling between various sets of display information.

A display 806 is coupled to the processor 802 via a display driver system 808. The display 806 can be a color liquid crystal display (LCD), plasma display, touch screen display or the like. In one example, the display 806 is a touch screen display. The display 806 functions to present data, graphics, or other information content. Additionally, the display 806 can display a variety of functions that control the execution of the device 800. For example, in a touch screen example, the display 806 can display touch selection buttons.

Power can be provided to the processor 802 and other components forming the hand-held device 800 by an onboard power system 810 (e.g., a battery pack). In the event that the power system 810 fails or becomes disconnected from the device 800, a supplemental power source 812 can be employed to provide power to the processor 802 (and other components (e.g., sensors)) and to charge the onboard power system 810. The processor 802 of the device 800 can induce a sleep mode to reduce the current draw upon detection of an anticipated power failure. It is to be appreciated that an additional power source (not shown) can be employed in addition to the power source 810. For example, the additional power source (not shown) can be employed to power the monitoring portion of the handheld device whereas power source 810 can be employed to power the core functionality of the device 800.

The device 800 includes a communication subsystem 814 that includes a data communication port 816, which is employed to interface the processor 802 with a remote computer, server, service, or the like. The port 816 can include at least one of Universal Serial Bus (USB) and IEEE 1394 serial communications capabilities. Other technologies can also be included, but, are not limited to, for example, infrared communication utilizing an infrared data port, Bluetooth™, etc.

The device 800 can also include a radio frequency (RF) transceiver section 818 in operative communication with the processor 802. The RF section 818 includes an RF receiver 820, which receives RF signals from a remote device via an antenna 822 and can demodulate the signal to obtain digital information modulated therein. The RF section 818 also includes an RF transmitter 824 for transmitting information to a remote device, for example, in response to manual user input via a user input (e.g., a keypad) and/or sensor component 826 or automatically in response to the completion of a sensor reading or other predetermined and programmed criteria.

The transceiver section 818 facilitates communication with a transponder system, for example, either passive or active, that is in use with physiological and/or environmental monitoring components. The processor 802 signals (or pulses) the remote transponder system via the transceiver 818, and detects the return signal in order to read the contents of the detected information. In one implementation, the RF section 818 further facilitates telephone communications using the device 800. In furtherance thereof, an audio I/O section 828 is provided as controlled by the processor 802 to process voice input from a microphone (or similar audio input device) and audio output signals (from a speaker or similar audio output device).

In another implementation, the device 800 can provide speech recognition 830 capabilities such that when the device 800 is used as a voice activated device, the processor 802 can facilitate high-speed conversion of the voice signals into text or operative commands. For example, the converted voice signals can be used to control the device 800 in lieu of using manual entry via the keypad 826.

Other devices such as a global positioning engine 832, an image capture system 834, health monitor component (e.g., sensor) 836 and multi-language component 838 can be provided within the housing of the device 800 to affect functionality described supra. For example, the image capture system 834 can be employed in connection with recreating a health-related event by capturing images corresponding to a health related event.

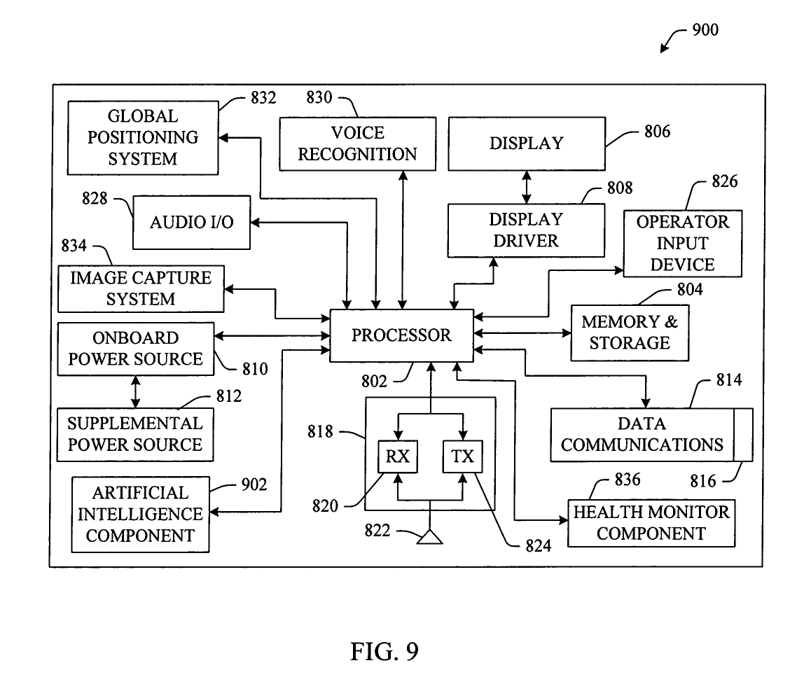

FIG. 9 illustrates a system 900 that employs artificial intelligence (AI) component 902 which facilitates automating one or more features in accordance with the subject invention. The subject invention (e.g., with respect to monitoring, sensing, communicating, . . . ) can employ various AI-based schemes for carrying out various aspects thereof. For example, a process for determining to initiate establishment of a health state, for transferring a health state (or data) and/or for language determination, conversion and/or detection can be facilitated via an automatic classifier system and process.

A classifier is a function that maps an input attribute vector, x=(x1, x2, x3, x4, xn), to a class label class(x). A classifier can also output a confidence that the input belongs to a class, that is, f(x)=confidence(x, class(x)). Such classification can employ a probabilistic and/or statistical-based analysis (e.g., factoring into the analysis utilities and costs) to prognose or infer an action that a user desires to be automatically performed.

A support vector machine (SVM) is an example of a classifier that can be employed. The SVM operates by finding a hypersurface in the space of possible inputs that splits the triggering input events from the non-triggering events in an optimal way. Other classification approaches including but not limited to Naïve Bayes, Bayesian networks, decision trees, neural networks, fuzzy logic models, can be employed. Classification as used herein also is inclusive of statistical regression that is utilized to develop models of priority.

As will be readily appreciated from the subject specification, the subject invention can employ classifiers that are explicitly trained (e.g., via a generic training data) as well as implicitly trained by using methods of reinforcement learning (e.g., via observing user behavior, observing trends, receiving extrinsic information). Thus, the subject invention can be used to automatically learn and perform a number of functions, including but not limited to determining, according to a predetermined criteria, a present and/or target location, services to pool, when/if to pool resources, which language and/or translation to employ, etc.

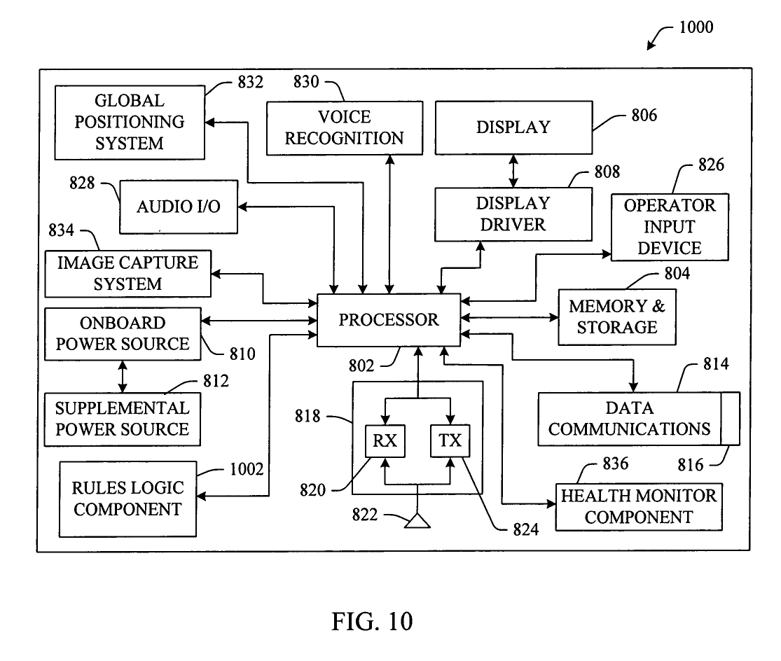

With reference now to FIG. 10, an alternate aspect of the invention is shown. More particularly, handheld device 1000 generally includes a rules-based logic component 1002. In accordance with this alternate aspect, an implementation scheme (e.g., rule) can be applied to define thresholds, initiate monitoring, facilitate communication, etc. By way of example, it will be appreciated that the rule-based implementation of FIG. 10 can automatically define criteria thresholds whereby an analyzer component or processor 802 can employ the thresholds to determine a health state and to effect an action.

In response thereto, the rule-based implementation can initiate communication and/or transmission of sensor readings and health states by employing a predefined and/or programmed rule(s) based upon any desired criteria (e.g., health history). It is to be understood and appreciated that a health state can refer to any data associated with a sensor reading. Further, a health state can be raw data received from a sensor as well as computed information based upon a compilation of factors and data. For example, a health state can refer to a relationship of data including physiological data, environmental data, statistical data, etc.

By way of further example, a user can establish a rule that can prompt a particular sensor monitoring. Accordingly, a rule-based decision logic can be employed to effect an action based upon the obtained and/or manipulated data. It will be appreciated that any of the specifications and/or functionality utilized in accordance with the subject invention can be programmed into a rule-based implementation scheme. It is also to be appreciated that this rules-based logic can be employed in addition to, or in place of, the AI reasoning components described with reference to FIG. 9.

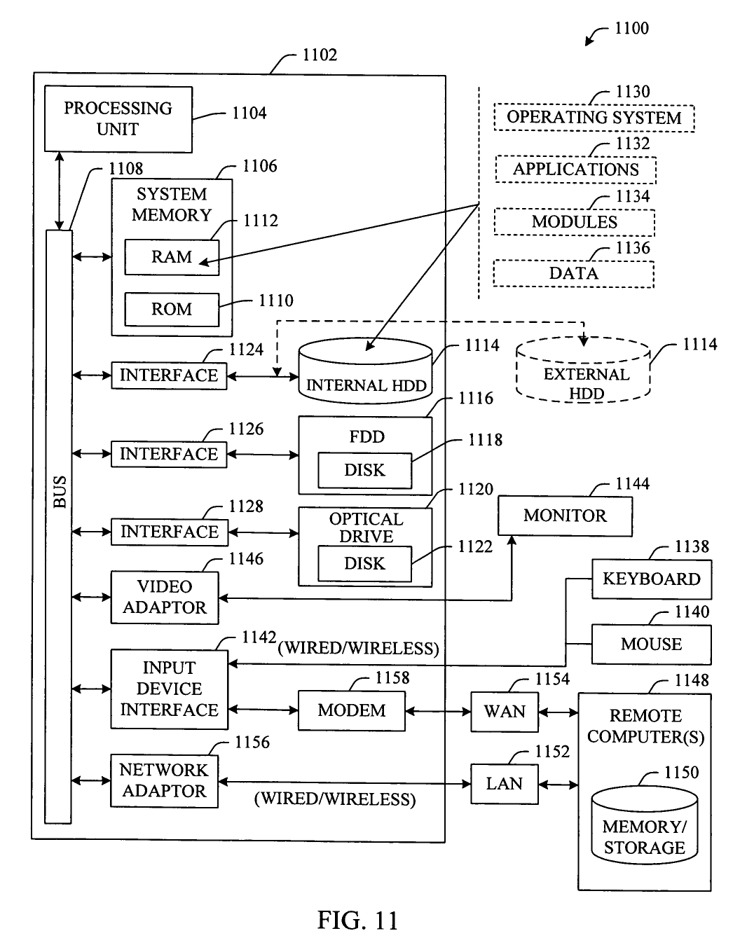

Referring now to FIG. 11, there is illustrated a block diagram of a computer operable to execute the disclosed architecture. More particularly, the block diagram of FIG. 11 can be employed in connection with the portable device and/or remote PC. In order to provide additional context for various aspects of the subject invention, FIG. 11 and the following discussion are intended to provide a brief, general description of a suitable computing environment 1100 in which the various aspects of the invention can be implemented. While the invention has been described above in the general context of computer-executable instructions that may run on one or more computers, those skilled in the art will recognize that the invention also can be implemented in combination with other program modules and/or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the invention may also be practiced in distributed computing environments where certain tasks are performed by remote processing devices that are linked through a communications network. In a distributed computing environment, program modules can be located in both local and remote memory storage devices.

A computer typically includes a variety of computer-readable media. Computer-readable media can be any available media that can be accessed by the computer and includes both volatile and nonvolatile media, removable and non-removable media. By way of example, and not limitation, computer-readable media can comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital video disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

With reference again to FIG. 11, the exemplary environment 1100 for implementing various aspects of the invention includes a computer 1102, the computer 1102 including a processing unit 1104, a system memory 1106 and a system bus 1108. The system bus 1108 couples system components including, but not limited to, the system memory 1106 to the processing unit 1104. The processing unit 1104 can be any of various commercially available processors. Dual microprocessors and other multi-processor architectures may also be employed as the processing unit 1104.

The system bus 1108 can be any of several types of bus structure that may further interconnect to a memory bus (with or without a memory controller), a peripheral bus, and a local bus using any of a variety of commercially available bus architectures. The system memory 1106 includes read-only memory (ROM) 1110 and random access memory (RAM) 1112. A basic input/output system (BIOS) is stored in a non-volatile memory 1110 such as ROM, EPROM, EEPROM, which BIOS contains the basic routines that help to transfer information between elements within the computer 1102, such as during start-up. The RAM 1112 can also include a high-speed RAM such as static RAM for caching data.

The computer 1102 further includes an internal hard disk drive (HDD) 1114 (e.g., EIDE, SATA), which internal hard disk drive 1114 may also be configured for external use in a suitable chassis (not shown), a magnetic floppy disk drive (FDD) 1116, (e.g., to read from or write to a removable diskette 1118) and an optical disk drive 1120, (e.g., reading a CD-ROM disk 1122 or, to read from or write to other high capacity optical media such as the DVD). The hard disk drive 1114, magnetic disk drive 1116 and optical disk drive 1120 can be connected to the system bus 1108 by a hard disk drive interface 1124, a magnetic disk drive interface 1126 and an optical drive interface 1128, respectively. The interface 1124 for external drive implementations includes at least one or both of Universal Serial Bus (USB) and IEEE 1394 interface technologies. Other external drive connection technologies are within contemplation of the subject invention.

The drives and their associated computer-readable media provide nonvolatile storage of data, data structures, computer-executable instructions, and so forth. For the computer 1102, the drives and media accommodate the storage of any data in a suitable digital format. Although the description of computer-readable media above refers to a HDD, a removable magnetic diskette, and a removable optical media such as a CD or DVD, it should be appreciated by those skilled in the art that other types of media which are readable by a computer, such as zip drives, magnetic cassettes, flash memory cards, cartridges, and the like, may also be used in the exemplary operating environment, and further, that any such media may contain computer-executable instructions for performing the methods of the invention.

A number of program modules can be stored in the drives and RAM 1112, including an operating system 1130, one or more application programs 1132, other program modules 1134 and program data 1136. All or portions of the operating system, applications, modules, and/or data can also be cached in the RAM 1112. It is appreciated that the invention can be implemented with various commercially available operating systems or combinations of operating systems.

A user can enter commands and information into the computer 1102 through one or more wired/wireless input devices, e.g., a keyboard 1138 and a pointing device, such as a mouse 1140. Other input devices (not shown) may include a microphone, an IR remote control, a joystick, a game pad, a stylus pen, touch screen, or the like. These and other input devices are often connected to the processing unit 1104 through an input device interface 1142 that is coupled to the system bus 1108, but can be connected by other interfaces, such as a parallel port, an IEEE 1394 serial port, a game port, a USB port, an IR interface, etc.

A monitor 1144 or other type of display device is also connected to the system bus 1108 via an interface, such as a video adapter 1146. In addition to the monitor 1144, a computer typically includes other peripheral output devices (not shown), such as speakers, printers, etc.

The computer 1102 may operate in a networked environment using logical connections via wired and/or wireless communications to one or more remote computers, such as a remote computer(s) 1148. The remote computer(s) 1148 can be a workstation, a server computer, a router, a personal computer, portable computer, microprocessor-based entertainment appliance, a peer device or other common network node, and typically includes many or all of the elements described relative to the computer 1102, although, for purposes of brevity, only a memory/storage device 1150 is illustrated. The logical connections depicted include wired/wireless connectivity to a local area network (LAN) 1152 and/or larger networks, e.g., a wide area network (WAN) 1154. Such LAN and WAN networking environments are commonplace in offices and companies, and facilitate enterprise-wide computer networks, such as intranets, all of which may connect to a global communications network, e.g., the Internet.

When used in a LAN networking environment, the computer 1102 is connected to the local network 1152 through a wired and/or wireless communication network interface or adapter 1156. The adaptor 1156 may facilitate wired or wireless communication to the LAN 1152, which may also include a wireless access point disposed thereon for communicating with the wireless adaptor 1156.

When used in a WAN networking environment, the computer 1102 can include a modem 1158, or is connected to a communications server on the WAN 1154, or has other means for establishing communications over the WAN 1154, such as by way of the Internet. The modem 1158, which can be internal or external and a wired or wireless device, is connected to the system bus 1108 via the serial port interface 1142. In a networked environment, program modules depicted relative to the computer 1102, or portions thereof, can be stored in the remote memory/storage device 1150. It will be appreciated that the network connections shown are exemplary and other means of establishing a communications link between the computers can be used.

The computer 1102 is operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi and Bluetooth™ wireless technologies. Thus, the communication can be a predefined structure as with a conventional network or simply an ad hoc communication between at least two devices.

Wi-Fi, or Wireless Fidelity, allows connection to the Internet from a couch at home, a bed in a hotel room, or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11 (a, b, g, etc.) to provide secure, reliable, fast wireless connectivity. A Wi-Fi network can be used to connect computers to each other, to the Internet, and to wired networks (which use IEEE 802.3 or Ethernet). Wi-Fi networks operate in the unlicensed 2.4 and 5 GHz radio bands, at an 11 Mbps (802.11a) or 54 Mbps (802.11b) data rate, for example, or with products that contain both bands (dual band), so the networks can provide real-world performance similar to the basic 10BaseT wired Ethernet networks used in many offices.

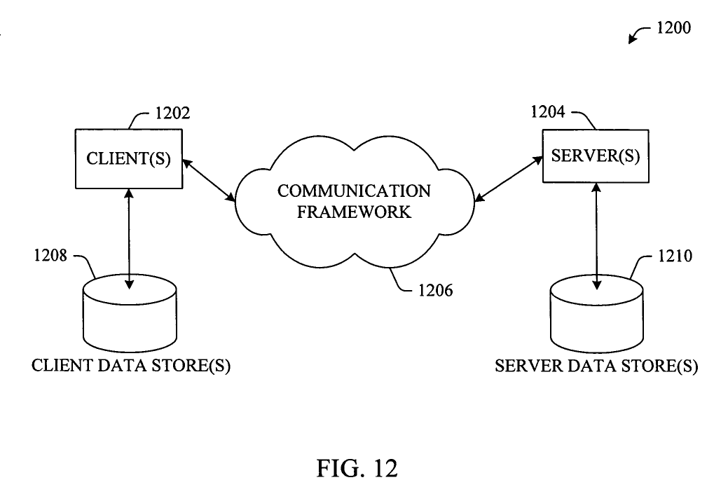

Referring now to FIG. 12, there is illustrated a schematic block diagram of an exemplary computing environment 1200 in accordance with the subject invention. As illustrated in FIG. 12, it is to be understood that the “client(s)” can be representative of a portable device and the “server(s)” can be representative of a remote PC or other remote server and/or host computer. As shown, the system 1200 includes one or more client(s) 1202. The client(s) 1202 can be hardware and/or software (e.g., threads, processes, computing devices). The client(s) 1202 can house cookie(s) and/or associated contextual information by employing the invention, for example.

The system 1200 also includes one or more server(s) 1204. The server(s) 1204 can also be hardware and/or software (e.g., threads, processes, computing devices). The servers 1204 can house threads to perform transformations by employing the invention, for example. One possible communication between a client 1202 and a server 1204 can be in the form of a data packet adapted to be transmitted between two or more computer processes. The data packet may include a cookie and/or associated contextual information, for example. The system 1200 includes a communication framework 1206 (e.g., a global communication network such as the Internet) that can be employed to facilitate communications between the client(s) 1202 and the server(s) 1204.

Communications can be facilitated via a wired (including optical fiber) and/or wireless technology. The client(s) 1202 are operatively connected to one or more client data store(s) 1208 that can be employed to store information local to the client(s) 1202 (e.g., cookie(s) and/or associated contextual information). Similarly, the server(s) 1204 are operatively connected to one or more server data store(s) 1210 that can be employed to store information local to the servers 1204.

What has been described above includes examples of the invention. It is, of course, not possible to describe every conceivable combination of components or methodologies for purposes of describing the subject invention, but one of ordinary skill in the art may recognize that many further combinations and permutations of the invention are possible. Accordingly, the invention is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A system that facilitates determining a physiological state of an individual, comprising:
a monitor component that obtains an input from at least one sensing component; and
an analyzer component that evaluates the input and determines the physiological state of the individual.

2. The system of claim 1, the at least one sensing component comprises at least one of an environmental sensor component and a physiological sensor component.

3. The system of claim 1, the at least one sensing component comprises an environmental sensor component and a physiological sensor component.

4. The system of claim 1, further comprising a communication component that facilitates an action based at least in part upon the evaluated input.

5. The system of claim 4, the action is communicating to a user at least one of the state of the individual, the state of the environment, and a recommended action based upon one or both of these states.

6. The system of claim 4, the action is remotely recording the input.

7. The system of claim 1, the monitor component is a removable expansion module included in a mobile device.

8. The system of claim 1, further comprising an artificial intelligence (AI) component that employs a reasoning analysis to infer an action to be automatically performed, the action comprises at least one of alerting the user, alerting/directing individuals near the user, alerting a pre-determined individual and alerting an emergency service.

9. A computer-readable medium having stored thereon computer-executable instructions for carrying out the system of claim 1.

10. A portable device that employs the system of claim 1.

11. A portable communications device that employs the system of claim 1.

12. A computer-implemented method of monitoring a health state of an individual, comprising:
receiving an input;
analyzing the input; and
generating a proactive health plan based at least in part on the analyzed input.

13. The computer-implemented method of claim 12, the input is a physiological-based input associated to the individual.

14. The computer-implemented method of claim 12, the input is an environmental input based at least in part on a proximate location of the individual.

15. The computer-implemented method of claim 12, further comprising communicating the health state to a medical service.

16. The computer-implemented method of claim 15, further comprising recording a plurality of health states of the individual.

17. The computer-implemented method of claim 16, further comprising determining a location of the individual and communicating the location to the medical service.

18. A system that facilitates generating a health history of an individual, comprising:
means for receiving a plurality of inputs, the inputs are physiological inputs and environmental inputs;
means for analyzing each of the plurality of inputs to determine a plurality of corresponding health states; and
means for recording the plurality of health states to generate the health history.

19. The system of claim 18, further comprising means for generating a health plan based at least in part on the health history.

20. The system of claim 19, further comprising means for alerting a disparate individual based at least in part on each of the analyzed inputs.
